(19) United States
(12) Patent Application Publication
(10) Pub. No.: US 2017/0095539 A1
(43) Pub. Date: Apr. 6, 2017

(54) Composition for Prevention and Treatment of Joint Pain and the Method of Preparation Thereof

(71) Applicant: THE BEAUTY NATION PTE. LTD., Singapore (SG)

(72) Inventor: SEE LENG TAN, SINGAPORE (SG)

(21) Appl. No.: 15/315,963

(22) PCT Filed: Jun. 2, 2015
(86) PCT No.: PCT/SG2015/050138

§ 371 Date: Dec. 2, 2016

(30) Foreign Application Priority Data
Jun. 2, 2014 (SG) 10201402819W

(51) Int. Cl. A61K 38/39; A61K 36/9066; A61K 31/10; A61K 36/126; A61K 36/185; A61K 31/737; A61K 36/296; A61K 36/746

(52) U.S. Cl. CPC A61K 38/39; A61K 36/296; A61K 36/9066; A61K 36/746; A61K 36/126; A61K 36/185; A61K 31/737; A61K 31/10

(57) ABSTRACT

The present invention discloses a herbal composition for prevention and treatment of joint pain, the method of preparation thereof, and its application thereof. The composition relieves osteoarthritis pain and substantially treats osteoarthritis. It can be used for the prevention and treatment of osteoarthritis of various joints, including the knee joint, hip joint, wrist joint, spine joint, shoulder joint, and ankle joint. The composition disclosed herein can be formulated into tablet, capsule, dispersible tablet, chewable tablet, effervescent tablet, and buccal tablet. The composition comprises a mixture of glucosamine sulfate 2KCl, chondroitin sulfate, methyl-sulfonyl-methane (MSM), and any one or a combination of Sambucus williamsii Hance extract and Drynaria fortunei (Kunze) J. Sm. extract. Further, the mixture may comprise any one or a combination of type II collagen, Morinda officinalis root extract, Epimedium grandiflorum extract, and turmeric root extract. Preferably, the mixture is sieved and formulated into a dosage form with 0.10 g to 1.50 g of the mixture per dose. As compared to the prior art, the present invention offers advantages such as simple processing method, explicit functional components, and minimal side effects.

### FIELD OF INVENTION

The present invention relates to a composition for prevention and treatment of joint pain, and the method of preparing the composition. More particularly, it relates to a composition for treatment of joint pain comprising medicinal herbs and the method of preparation thereof.

### BACKGROUND OF THE INVENTION

Osteoarthritis (OA) is a type of chronic degenerative joint disease which affects the health of elderly. It is a common, frequently-occurring disease which affects people regardless of ethnicity and geographical differences. It is characterized by degeneration of articular cartilage, continual wearing of articular cartilage, and hyperosteogeny (excessive bone development). Onset of this disease can occur at the age of 20. The occurrence of this disease is hard to be discovered at initial onset as it is often asymptomatic. Clinically, osteoarthritis of the knee is the most common type of osteoarthritis. The occurrence of osteoarthritis is increasing with the increased population of elderly in the world. It greatly affects the quality of life of the elderly. The clinical manifestations of osteoarthritis include joint pain, joint swelling, and joint dysfunction. Key pathological changes of osteoarthritis include degeneration of articular cartilage, remodeling of subchondral bone, and change of synovium, which eventually lead to the degradation of cartilage matrix, death of cartilage cell, and destruction of the structural integrity of joints. There are various causes of osteoarthritis, including aging, wearing of cartilage, obesity, biochemical factors, and genetic factors. These factors inhibit the synthesis of cartilage-matrix proteoglycan and promote the degradation of proteoglycan, hyaluronic acid, and collagen.

Treatments for knee pain mainly consist of physiotherapy, modern western medication, and traditional Chinese medication. However, physiotherapy can only relieve the osteoarthritis pain, thus it serves as a complementary treatment for osteoarthritis. Further, modern western medication easily causes irritation of the gastrointestinal tract and has negative impact on the patient's health. Traditional Chinese medication takes a long time to work and has slow and non-obvious effects of treatment for osteoarthritis, hence it is not widely accepted by patients.

### SUMMARY OF THE INVENTION

One of the objects of the invention is to provide a composition for prevention of joint pain, especially joint pain caused by osteoarthritis.

Another object of the invention is to provide a composition for treatment of joint pain, especially joint pain caused by osteoarthritis.

At least one of the preceding objects is met, in whole or in part, by the invention, in which the embodiment of the invention describes a composition for prevention and treatment of joint pain comprising a mixture of glucosamine sulfate 2KCl, chondroitin sulfate, methyl-sulfonyl-methane (MSM), and any one or a combination of Sambucus williamsii Hance extract and Drynaria fortunei (Kunze) J. Sm. extract.

In one preferred embodiment of the invention, the weight percentage in relative to the total composition of glucosamine sulfate 2KCl is 10% to 50%; chondroitin sulfate is 10% to 40%; methyl-sulfonyl-methane (MSM) is 5% to 40%; Sambucus williamsii Hance extract is 5% to 40%; and Drynaria fortunei (Kunze) J. Sm. extract is 5% to 40%.

In another preferred embodiment of the invention, the composition may further comprise any one or a combination of type II collagen, Morinda officinalis root extract, Epimedium grandiflorum extract, and turmeric root extract, wherein each of the components may present in the composition from 5% to 40% by weight of the total composition.

In a further preferred embodiment of the invention, the composition comprises a mixture of, by weight of the total composition, 10% to 50% of glucosamine sulfate 2KCl, 10% to 40% of chondroitin sulfate, 5% to 40% of methyl-sulfonyl-methane (MSM), 5% to 40% of type II collagen, 5% to 40% of Morinda officinalis root extract, 5% to 40% of Epimedium grandiflorum extract, 5% to 40% of Sambucus williamsii Hance extract, 5% to 40% of Drynaria fortunei (Kunze) J. Sm. extract, and 5% to 40% of turmeric root extract.

More specifically, the composition comprises a mixture of, by weight of the total composition, 35% of glucosamine sulfate 2KCl, 20% of chondroitin sulfate, 10% of methyl-sulfonyl-methane (MSM), 10% of type II collagen, 5% of Morinda officinalis root extract, 5% of Epimedium grandiflorum extract, 5% of Sambucus williamsii Hance extract, 5% of Drynaria fortunei (Kunze) J. Sm. extract, and 5% of turmeric root extract.

In accordance with the preferred embodiment of the invention, the mixture is sieved and fabricated into a dosage form by performing granulation, filling or tableting process. Preferably, the dosage form comprises 0.10 g to 1.50 g of the mixture.

The dosage form includes, but not limited to, tablet, sugar-coated tablet, film-coated tablet, hard-shelled capsule, soft-shelled capsule, enteric-coated capsule, granule, pill, and powder.

### DETAILED DESCRIPTION OF THE INVENTION

It is to be understood that the present invention may be embodied in other specific forms and is not limited to the sole embodiment described herein. However, modification and equivalents of the disclosed concepts such as those which readily occur to one skilled in the art are intended to be included within the scope of the claims which are appended thereto.

The object of the present invention is to provide a composition for prevention and treatment of joint pain, and the method of preparation thereof. Particularly, it has beneficial therapeutic effect in treatment of knee pain.

The composition embodied herein mainly comprises glucosamine sulfate 2KCl, chondroitin sulfate, methyl-sulfonyl-methane (MSM), type II collagen, Morinda officinalis root extract, Epimedium grandifloruin extract, Sambucus williamsii Hance extract, Drynaria fortunei (Kunze) J. Sm. extract, and turmeric root extract.

In the preferred embodiment of the present invention, the composition for prevention and treatment of joint pain comprises a mixture of glucosamine sulfate 2KCl, chondroitin sulfate, methyl-sulfonyl-methane (MSM), and any one or a combination of Sambucus williamsii Hance extract and Drynaria fortunei (Kunze) J. Sm. extract. In another preferred embodiment of the present invention, the mixture may further comprise any one or a combination of type II collagen, Morinda officinalis root extract, Epimedium grandiflorum extract, and turmeric root extract.

In the preferred embodiment of the present invention, the mixture comprises 10% to 50% of glucosamine sulfate 2KCl by weight of total composition. Glucosamine sulfate 2KCl is able to stimulate regeneration of cartilage structure. It can also promote cartilage metabolism in order to prevent the occurrence of cartilage structure degradation. Further, it aids in the repairing of worn joint tissues, thereby reducing joint pain and joint swelling, and improving the flexibility of the joint. Besides, the combined use of glucosamine sulfate 2KCl and chondroitin sulfate has synergistic effect in treating joint pain.

Preferably, chondroitin sulfate constitutes 10% to 40% by weight of total composition. Chondroitin sulfate (CS) is found in abundance in cartilage. It allows cartilage to provide joint cushioning and lubrication by absorbing water into the cartilage. It also promotes regeneration of cartilage via synthesis of collagen, which is an essential protein in cartilage. Therefore, the combined use of glucosamine sulfate 2KCl and chondroitin sulfate can alleviate the symptoms of osteoarthritis, normalize the metabolism of cartilage, improve the mobility of joint, and increase bone density and bone rigidity. Besides, chondroitin sulfate can reduce serum uric acid levels, thereby preventing the occurrence of gout, treating gout, and alleviating the symptoms associated with gout.

In accordance with the preferred embodiment of the invention, the mixture comprises 5% to 40% of methyl-sulfonyl-methane (MSM) by weight of total composition. Methyl-sulfonyl-methane (MSM) is a natural analgesic agent as it can reduce inflammation and pain. It is relatively safer than commercially available analgesic. MSM can reduce joint inflammation and protect joint from damage. MSM is necessary for the synthesis of collagen, which is the major component of cartilage. Clinical studies show that MSM can help to improve the quality of life of patients with osteoarthritis and may alleviate certain symptoms of osteoarthritis such as joint pain and joint stiffness.

Pursuant to the preferred embodiment of the invention, the mixture may comprise 5% to 40% of type II collagen by weight of total composition. Type II collagen is the major component of articular cartilage, epiphyseal cartilage, and trabecular bone. Type II collagen constitutes 70% to 86% of the bone. Further, collagen is an important component of skin and muscles. It helps to maintain bone rigidity, muscle coordination, and skin elasticity.

The composition described herein comprises Morinda officinalis root extract, Epimedium grandiflorum extract, Sambucus williamsii Hance extract, Drynaria fortunei (Kunze) J. Sm. extract, and turmeric root extract. These extracts synergistically provide good therapeutic effects in the disclosed composition.

Morinda officinalis root extract may present in the mixture at 5% to 40% by weight of total composition. Morinda officinalis root is traditionally used to improve bone and liver health, reduce inflammation (swelling and pain), treat bone diseases such as osteoporosis and osteonecrosis of femoral head, treat arthritis, promote bone synthesis, reduce osteolysis, and prevent occurrence of bone fractures due to osteoporosis.

The mixture may comprise 5% to 40% of Epimedium grandiflorum extract by weight of total composition. Epimedium grandiflorum is able to invigorate kidney and strengthen bones. Its active ingredients can promote the proliferation and differentiation of bone cells (i.e. osteoblast), so E. grandifiorum can be used to prevent and treat gouty arthritis, including acute gouty arthritis and chronic gouty arthritis. Further, E. grandiflorum is beneficial to the cardiovascular system, central nervous system, circulatory system, and immune system. It also possesses anti-inflammatory, anti-osteoporotic, and anti-aging activity.

Preferably, the mixture comprises 5% to 40% of Sambucus williamsii Hance extract by weight of total composition. Sambucus williamsii Hance can reduce inflammation and relieve pain. It can be used to treat rheumatoid arthritis, bruises, fractures, gout, Kashin-Beck disease, and edema. In Europe, people have been using it to treat arthritis, asthma, cold, constipation, and related diseases.

In the preferred embodiment of the invention, the mixture comprises 5% to 40% of Drynaria fortunei (Kunze) J. Sm. extract by weight of total composition. Drynaria fortunei (Kunze) J. Sm. is able to improve kidney and liver health, and relieve pain. It can be used to treat weak kidneys, back pain, tinnitus, hearing impairment, loose teeth, and injuries such as sprain and strain. Besides, it can also lower post-meal blood sugar level and blood uric acid level, and treat diseases such as impaired glucose tolerance, diabetes, obesity, hyperuricemia, and osteoporosis. Research shows that D. fortunei can promote calcium absorption and proteoglycans synthesis, increase levels of calcium and phosphorus in blood, support ossification and fracture healing, and delay degeneration of bone cells.

In accordance with the preferred embodiment of the invention, the mixture may comprise 5% to 40% of turmeric root by weight of total composition. Turmeric root can be used to relieve chest pain, rheumatoid arthritis pain in the arms and shoulders, and pain due to bruising and swelling of the joint. Research reveals that turmeric root prevents gout and inhibits the function of α-glucosaminidase. Turmeric root is able to reduce serum uric acid level by inhibiting synthesis of uric acid and promoting excretion of uric acid.

More preferably, the composition embodied herein comprises a mixture of, by weight of total composition, 10% to 50% of glucosamine sulfate 2KCl, 10% to 40% of chondroitin sulfate, 5% to 40% of methyl-sulfonyl-methane (MSM), 5% to 40% of type II collagen, 5% to 40% of Morinda officinalis root extract, 5% to 40% of

Epimedium grandiflorm extract, 5% to 40% of Sambucus williamsii Hance extract, 5% to 40% of Drynaria fortunei (Kunze) J. Sm. extract, and 5% to 40% of turmeric root extract.

Most preferably, the composition comprises a mixture of, by weight of total composition, 35% of glucosamine sulfate 2KCl, 20% of chondroitin sulfate, 10% of methyl-sulfonyl-methane (MSM), 10% of type II collagen, 5% of Morinda officinalis root extract, 5% of Epimedium grandiflorum extract, 5% of Sambucus williamsii Hance extract, 5% of Drynaria fortunei (Kunze) J. Sm. extract, and 5% of turmeric root extract.

Pursuant to the preferred embodiment of the invention, the mixture is sieved and fabricated into a dosage form by performing granulation, filling or tableting processes. Particularly, the dosage form comprises 0.10 g to 1.50 g of the mixture. The dosage form includes, but not limited to, tablet, sugar-coated tablet, film-coated tablet, hard-shelled capsule, soft-shelled capsule, enteric-coated capsule, granule, pill, and powder.

The recommended intake is two to three times daily, 2 dosages each time. Preferably, it is ingested with warm water.

The composition embodied herein is suitable for use by people with arthritis (e.g. gouty arthritis) and people experiencing joint pain, joint swelling, joint stiffness, joint cracking, and joint immobility. Further, it is also suitable for use by people with high amount of physical activities, people who are prone to joint sprain, people who stay in a position for long period, people with repetitive strain injuries (i.e. wrist sprain and neck sprain), middle-aged and older age groups with bone degeneration issues, and people who are in need for improvement in bone density and bone rigidity. The disclosed composition is beneficial to people with joint and bone diseases, for example and without limitation, hyperosteogeny, osteoporosis, rheumatoid arthritis, gout, joint strain, spinal disc herniation, and adhesive capsulitis (frozen shoulder).

The present invention provides a composition for prevention and treatment of joint pain. The composition as set forth in foregoing description helps to repair cartilage matrix, alleviate exercise-related or work-related joint pain, soothe joint pain, joint stiffness, joint swelling, and back pain, repair injured joints, lessen joint inflammation and pain, reduce damage to cartilage, and relieve frozen shoulder, tennis elbow (lateral epicondylitis), golfer's elbow (medial epicondylitis), and sprains. Further, it also prevents uric acid-induced gouty arthritis. Specifically, the composition embodied herein possesses anti-inflammatory, analgesic, and anti-gout activity.

Particularly, the composition embodied herein has the following benefits: (i) accelerating regeneration and repair of cartilage; (ii) providing major components required in the repair of cartilage, tendon, and ligament; (iii) reducing strain on cartilage, bones, and adjacent joint structures and supporting regeneration of joint tissues; (iv) stimulating the repair and regeneration of elastic cartilage, thereby preventing loss of elasticity in cartilage; (v) soothing inflammation, enlargement, swelling, and atrophy of joint; (vi) preventing loss of elasticity and water in cartilage, and preventing cartilage from becoming brittle; (vii) controlling balance of synovial fluid secretion to prevent atrophy of joint; (viii) easing inflammation of ligaments and muscles around the joints; (ix) alleviating inflammation, pain, and swelling associated with osteoarthritis and rheumatoid arthritis; and (x) preventing and slowing down joint damage, joint deformation, and joint calcification.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1-25. (canceled)

26. A composition for prevention and treatment of joint pain, comprising:
a mixture of glucosamine sulfate 2KCl, chondroitin sulfate, methyl-sulfonyl-methane (MSM), type II collagen, and any one or a combination of Sambucus williamsii Hance extract and Drynaria fortunei (Kunze) J. Sm. extract.

27. The composition according to claim 26, wherein the mixture further comprises Morinda officinalis root extract.

28. The composition according to claim 26, wherein the mixture further comprises Epimedium grandiflorum extract.

29. The composition according to claim 26, wherein the mixture further comprises turmeric root extract.

30. The composition according to claim 26, wherein the mixture comprises 10% to 50% of glucosamine sulfate 2KCl by weight of total composition.

31. The composition according to claim 26, wherein the mixture comprises 10% to 40% of chondroitin sulfate by weight of total composition.

32. The composition according to claim 26, wherein the mixture comprises 5% to 40% of methyl-sulfonyl-methane (MSM) by weight of total composition.

33. The composition according to claim 26, wherein the mixture comprises 5% to 40% of Sambucus williamsii Hance extract by weight of the composition.

34. The composition according to claim 26, wherein the mixture comprises 5% to 40% of Drynaria fortunei (Kunze) J. Sm. extract by weight of the composition.

35. The composition according to claim 26, wherein the mixture comprises 5% to 40% of type II collagen by weight of the composition.

36. The composition according to claim 27, wherein the mixture comprises 5% to 40% of Morinda officinalis root extract by weight of the composition.

37. The composition according to claim 28, wherein the mixture comprises 5% to 40% of Epimedium grandiflorum extract by weight of the composition.

38. The composition according to claim 29, wherein the mixture comprises 5% to 40% of turmeric root extract by weight of the composition.

39. The composition according to claim 26, wherein the mixture is sieved and fabricated into a dosage form by performing granulation, filling or tableting process.

40. The composition according to claim 39, wherein the dosage form comprises 0.10 grams to 1.50 grams of the mixture.

41. The composition according to claim 40, wherein the dosage form is tablet, sugar-coated tablet, film-coated tablet, hard-shelled capsule, soft-shelled capsule, enteric-coated capsule, granule, pill, or powder.

42. A composition for prevention and treatment of joint pain, comprising:
a mixture of, by weight of the total composition, 10% to 50% of glucosamine sulfate 2KCl, 10% to 40% of chondroitin sulfate, 5% to 40% of methyl-sulfonyl-methane (MSM), 5% to 40% of type II collagen, 5% to 40% of Morinda officinalis root extract, 5% to 40% of Epimedium grandiflorum extract, 5% to 40% of Sambucus williamsii Hance extract, 5% to 40% of Drynaria fortunei (Kunze) J. Sm. extract, and 5% to 40% of turmeric root extract.

43. The composition according to claim 42, wherein the mixture is sieved and fabricated into a dosage form by performing granulation, filling or tableting process.

44. The composition according to claim 43, wherein the dosage form comprises 0.10 grams to 1.50 grams of the mixture.

45. The composition according to claim 44, wherein the dosage form is tablet, sugar-coated tablet, film-coated tablet, hard-shelled capsule, soft-shelled capsule, enteric-coated capsule, granule, pill, or powder.

46. A composition for prevention and treatment of joint pain, comprising:
a mixture of, by weight of the composition, 35% of glucosamine sulfate 2KCl, 20% of chondroitin sulfate, 10% of methyl-sulfonyl-methane (MSM), 10% of type II collagen, 5% of Morinda officinalis root extract, 5% of Epimedium grandiflorum extract, 5% of Sambucus williamsii Hance extract, 5% of Drynaria fortunei (Kunze) J. Sm. extract, and 5% of turmeric root extract.

47. The composition according to claim 46, wherein the mixture is sieved and fabricated into a dosage form by performing granulation, filling and tableting processes.

48. The composition according to claim 47, wherein the dosage form comprises 0.10 grams to 1.50 grams of the mixture.

49. The composition according to claim 48, wherein the dosage form is tablet, sugar-coated tablet, film-coated tablet, hard-shelled capsule, soft-shelled capsule, enteric-coated capsule, granule, pill, or powder.
